(19) United States
(12) Patent Application Publication
(10) Pub. No.: US 2007/0100457 A1
(43) Pub. Date: May 3, 2007

(54) Paramagnetic liquid interface

(76) Inventor: Edward R. Hyde JR., Turlock, CA (US)

Correspondence Address: Thomas M. Freiburger, P.O. Box 1026, Tiburon, CA, 94920, US

(21) Appl. No.: 11/073,125

(22) Filed: Mar. 4, 2005

Related U.S. Application Data
(60) Provisional application No. 60/521,183, filed on Mar. 4, 2004.
(60) Provisional application No. 60/521,657, filed on Jun. 12, 2004.

(51) Int. Cl. A61F 2/30

(52) U.S. Cl. 623/18.12

(57) ABSTRACT

Natural joint interfaces wear out and/or are damaged causing pain and disability. They can be currently replaced by artificial surfaces made of materials or in the near future by magnetic fields. They would benefit from a PVES (paramagnetic visco-viscoelastic supplement) that can replace or augment natural joint interfaces or augment total joint replacements. Joint replacement components can be modular and would benefit from a PVES to decrease wear and damp impact between modular parts of a single component. The PVES is a dynamic interface allowing components to be less rigid. Energy transmission is reduced. PVES can act as an interface between natural damaged joint surfaces obviating the need for classic total joint replacement or between the surfaces of artificial joint components to improve or supplement their function. These PVES can be controlled by magnetic fields with respect to their location, physical properties, loads, etc. PVES are typically made of paramagnetic ions and a substrate molecule. One such PVES can be made of gadolinium ions and hyaluronic acid to form gadolinium hyaluronate.

This application claims benefit from U.S. provisional applications No. 60/521,183, filed Mar. 4, 2004, and 60/521,657, filed Jun. 12, 2004.

### BACKGROUND AND SUMMARY OF THE INVENTION

This invention concerns treatment of joints of humans or animals using therapeutic liquids, and the retention of the liquid at a site using a magnetic field.

Total joint replacement (TJR) has been accepted as the preferred treatment for end stage arthritis. TJR requires the removal of damaged cartilage and bone and the substitution of components made of biocompatible materials. Currently the materials used in TJRs are metals, ceramics and plastics. These materials can also have surface treatments and enhancements. Metals that are currently used are stainless steel CoCr, titanium, etc. Plastics are currently polyethylenes. Ceramics can be alumina, etc.

Components are placed on one or both sides of the joint and these articulate with the opposing natural joint surface or another component of a TJR, to reproduce some of the functions of a normal joint. The most widely used material combinations are metal (cobalt-chrome) CoCr on polyethylene (ultra high molecular weight polyethylene) UHMWPE. There are also metal/metal and ceramic/ceramic and mixed combinations such as ceramic/UHMWPE.

During the complex motions of a joint the surfaces interact in adhesion, abrasion, impact, etc. These interactions lead to wear and loosening of the TJR components or further arthritic changes of the joint itself.

Lubrication between component surfaces is by fluids naturally produced by the joint. This lubrication reduces some of the inherent friction and wear characteristics and damps energy transmission. Wetting of surfaces is also important to natural joint function to provide smooth transition of energy into motion.

A supplemental paramagnetic liquid or paramagnetic visco-viscoelastic supplement (PVES) that would act as a fluid interface, lubricant or rheologic agent and would stay at the contact and peri-contact points between the components of the joint would decrease wear and would tend to damp energy transmission. The term paramagnetic as used herein refers to a material that is affected by a magnetic field. Visco-viscoelastic means viscous or viscoelastic.

Injectable viscous biocompatible materials such as hyaluronans (hyaluronic acid) have been used as a rheologic material in natural damaged joints designed to augment natural joint fluid in an effort to decelerate the progression of arthritis, especially osteoarthritis.

Supplemental hyaluronans are typically dispersed throughout the joint by the motion so very little is actually localized at the joint surfaces to act as a rheologic agent or lubricant. Also hyaluronans are absorbed quickly. The joint synovial lining produces new hyaluronic acid but composition characteristics can vary, such as molecular weight, etc. affecting the functionality. Supplemental high molecular weight hyaluronans are thought to augment low quality hyaluronans produced by the synovium affected by age or disease.

Joints can include all animal and mammalian anatomic joints of any classification. These joints include all joints of appendages, limbs and axial skeleton. This includes major joints such as the hip and knee, etc., small joints such as TMJ, shoulder, elbow, wrist, finger, etc. and articulations of the spine including spinal discs, facet joints, etc.

TJR components can be modular. A fluid interface between parts allows the individual parts to be less constrained. It also improves the function of fully constrained component parts that still demonstrate motion, (i.e. tibial insert and tibial tray - - - there is backside motion even though the components are meant to be locked together).

Paramagnetic visco-viscoelastic supplements (PVES) can act as an interface between natural damaged joint surfaces obviating the need for classic total joint replacement or between the surfaces of artificial joint components to improve or supplement their function.

Concentrating and maintaining the PVES or fluid interface between damaged natural joints or total joint replacement components, or between modular component parts or between natural components and replacement components, as well as concentrating the PVES with structural and/or therapeutic materials at a fracture site and keeping them from dispersing, comprises the principal scope of this invention. The invention's scope includes concentrating any therapeutic material at a body site.

Certain materials, compounds and elements, including ions, exhibit the property that they can be influenced by magnetic fields. The elements can be classed as ferromagnetic, paramagnetic and diamagnetic, depending on the way a magnetic field affects the material. Some materials can retain a magnetic field after it is removed and in effect become permanent magnets. The ability to retain a magnetic field is referred to as coercivity of the material.

Iron (Fe), cobalt (Co) and nickel (Ni) are ferromagnetic as are the lanthanide metals also known as rare earth metals. Gadolinium, samarium and neodymium are the most familiar rare earth elements. There are fifteen rare earth metals. They have different magnetic moments and are therefore affected by magnetic fields to different extents. Gadolinium is frequently used as a contrast agent for MRI studies.

Attaching rare earth ions, particles or compounds or other paramagnetic ions or compounds to other materials allows the compounds to be influenced by a magnetic field. Attachment can be via chemical bonds, intermediate molecules, or other means. The bond can be to a metal ion, or to a metal compound, through an intermediary molecule. Alternatively, paramagnetic particles can be retained in suspension in a carrier liquid.

Rare earth particles, compounds, ions (or other ions, particles or compounds influenced by magnetic fields) attached to organic or inorganic molecules or compounds allow the new compounds to be controlled or affected by magnets and/or magnetic fields.

Sodium hyaluronate or hyaluronic acid is a naturally formed substance. Sodium (Na) can be replaced, for example, by a rare earth ion such as gadolinium or neodymium. Gadolinium hyaluronate or neodymium hyaluronate can be produced by reactions and catalysts that exchange Na ions for Gd or other rare earth ions. Paramagnetic ions can be added to molecules by multiplicity of reactions or intermediate substances, well known in the field of organo-metallic chemistry.

The human body produces hyaluronic acid naturally. In a joint it exists as a viscous liquid. It is produced in joints and has mechanical and metabolic properties.

Gadolinium hyaluronate (rare earth hyaluronate or paramagnetic ion hyaluronate) is likewise hydrophilic and forms a very viscous liquid. Other viscous or viscoelastic supplements (VES), natural or synthetic, can be used rather than hyaluronate. The paramagnetic VES (PVES) liquid, however, will also migrate toward a magnet or a magnetic field source. It will collect in an area of influence of the magnetic field. When it is dispersed by mechanical interactions it will re-accumulate in the desired area, controlled by the magnetic field. The PVES will also form an accumulation with a thickness or height depending on the field strength, the density of the paramagnetic ions and magnetic moment of the ion. This provides stored potential energy to the PVES. The molecules with paramagnetic ions arrange in an orderly fashion under the influence of a magnetic field. The individual PVES molecules influence the PVES molecules near them. This accumulation of a magneto-rheologic liquid also exerts a pressure between two surfaces when the PVES is between them. The thickness of the thin-film layer is thicker at the greatest magnetic field concentration. The viscosity can also be altered by magneto-rheological influences of the magnetic field and liquid in addition to concentration and re-accumulation effects. Other chemical processes such as cross linking can increase the viscosity of the hyaluronate substrate or the VES.

Current HAS (hyaluronic acid supplements) are found to be absorbed from the knee in one to three days. HA with ferromagnetic or paramagnetic ions, pursuant to the invention, will resist re-absorption due to the influence of a magnetic field. The magnetic field can be generated by an internal or external source. The magnetic source can be from magnetic material (permanent magnets), electromagnets or magnetic induction or sources of external magnetic fields.

Any particle influenced by a magnetic field (paramagnetic) can be used (i.e. iron (Fe), rare earths). Any of the rare earths can be used. The difference in magnetic moment between different ions can be used to influence the molecule, and the material can be selected for specific properties.

Rare earths are weak to strong bases. They have different ionization potentials. The different properties can be used to develop chemical reactions and different characteristics for the fluids formed from these compounds.

Other organic molecules can be modified by the addition of paramagnetic ions. Natural and synthetic compounds can be modified by paramagnetic ions. Other hydrophilic molecules, natural or synthetic, can be used.

The PVES or fluid interface can be removed from a joint by removing the magnetic field source or applying a stronger magnetic source to the joint to move the material sufficiently far from an internal magnetic source. Removing or cycling the secondary magnetic source will allow the molecules to be collected or reabsorbed and cleared.

Additionally a magnetic source tipped needle or catheter can be introduced into the joint to collect the paramagnetic material. Irrigation can be used to flush the joint after the paramagnetic molecules have been moved away from the internal magnetic source by stronger magnetic fields. A magnetic separator can be used to assist the collection of the PVES.

Hyaluronic acid supplements (HAS), i.e. sodium hyaluronate, have been found to relieve pain in arthritic joints. Currently HAS is used most frequently in the knee to relieve symptoms for extended periods of time. The exact mechanism is unknown. There appears to be a pharmacological as well as a rheological or lubricating effect. It is well known, however, that the HAS is completely cleared from the joint in one to three days.

The addition of paramagnetic ions to a HAS or any alternative visco-viscoelastic supplement (VES) will allow the VES to be affected by a magnetic field. The magnetic field can be used to localize and concentrate the VES. The magnetic field also introduces energy into the VES that produces a force capable of doing work. This force resists the disbursement of the VES by mechanical forces. The magnetic field can also have a magneto-rheological effect on the VES, changing the viscosity. The rheological properties of the VES can be significantly enhanced by an applied field. The duration of the pharmacological properties of HAS will also be enhanced. The safety of the HAS will not be altered.

Any viscous supplement that is biocompatible can be used, natural or synthetic (i.e. hydrogel, etc.). The VES can be used in any joint, natural or artificial.

The fluid carrying paramagnetic ions or particles need not be rheologic for many applications. For example, a fluid that can bond to or has an affinity for a therapeutic substance (e.g. analgesic, antibiotic, pharmacological or radiopharmacological), or a fluid that itself is therapeutic, can be retained at a site where it is most effective, using an appropriately placed magnetic field.

The magnetic field source can be implanted and/or be external. A single magnet or magnetic source can be used or the source can be a paramagnetic material and exposed to a magnetic field or energy source. Paramagnetic VES can be used in conjunction with substantially repulsive magnetic arrays for certain applications.

The following patents have some relevance to the present invention: U.S. Pat. Nos. 6,482,436, 6,200,547, 5,705,195, 5,651,989, and 5,549,915.

### DESCRIPTION OF THE DRAWINGS

FIG. 1 shows a model of a fluid of the invention with magnetic material, surfactant and carrier fluid.

FIG. 1A shows a model of paramagnetic visco-viscoelastic supplement with paramagnetic ions & substrate.

FIG. 2 shows a paramagnetic ions attachment bond.

FIG. 3 shows a paramagnetic ions intermediary bond.

FIG. 4 shows a paramagnetic ions carrier fluid bond.

FIG. 5 shows a paramagnetic ions attachment affinity.

FIG. 6 shows a paramagnetic ions intermediary affinity.

FIG. 7 shows a paramagnetic ions carrier fluid affinity.

FIG. 8 shows a paramagnetic ions attachment bond with carrier fluid affinity.

FIG. 9 shows a paramagnetic ions intermediary bond with carrier fluid affinity.

FIG. 10 shows a paramagnetic ions attachment bond with carrier fluid bond.

FIG. 11 shows a magnetic material intermediary bond with carrier fluid bond.

FIG. 12 shows an attachment carrier fluid bond.

FIG. 13 shows an attachment carrier fluid affinity.

FIG. 14 shows a mammalian joint with PVES and plural magnetic sources.

FIG. 15 shows a mammalian joint with PVES and one magnetic source.

FIG. 16 shows a human knee joint with PVES and one magnetic source.

FIG. 17 shows a total knee replacement with PVES and magnetic sources.

FIG. 17A shows a cross section through FIG. 17.

FIG. 18 shows a total knee replacement insert/tibial tray interface of tibial component with PVES.

FIG. 18A shows a cross section through FIG. 18.

FIG. 18B shows a detail of portion of FIG. 18.

FIG. 19 shows a total knee replacement, indicating tibial tray/stem interface of tibial component with PVES or fluid interface.

FIGS. 19A and 19B are sectional and detail views from FIG. 19.

FIG. 20 shows a human hip joint with PVES and one magnetic source.

FIG. 21 shows a human radiocarpal joint with PVES and one magnetic source.

FIG. 22 shows a human first carpal metacarpal joint with PVES and magnetic source.

FIG. 23 shows a bone fracture with PVES and magnetic source.

### DESCRIPTION OF PREFERRED EMBODIMENTS

The following U.S. patents and published applications of the applicant include disclosures that may be relevant to procedures, structures, materials and compositions that are in part described herein, and all of these patents and published applications are incorporated herein by reference: U.S. Pat. Nos. 6,387,096, 6,599,321, 6,589,281, 6,716,249, and Publication Nos. 2003/0195633, 2003/0187510, 2002/0138149, 2002/0138148, 2002/0133153, 2002/0128651, and 2002/0111689.

A paramagnetic visco-viscoelastic supplement (PVES) can be directly introduced into an animal joint site as a liquid (having the property of flowing: consisting of particles that move freely among themselves, so as to give way with the slightest pressure) that can be controlled or activated by a magnetic field. Once introduced into an animal joint, the PVES works in combination with an applied or implanted source of magnetic field, to perform or alter mechanical work and/or maintain a PVES within the joint or fracture site for other therapeutic reasons.

The PVES can augment or substitute for the interface of a natural joint, or can augment artificial joint component interactions, or the fracture callus between fracture fragments or bone parts. The PVES can be a liquid, said of a material substance in that condition familiar as the normal condition of water, oil, alcohol, etc. in which its particles move freely over each other, so that its masses have no determined shape but do not tend to separate as do those of a gas: not solid nor gaseous. These include gels, semi-solids etc.

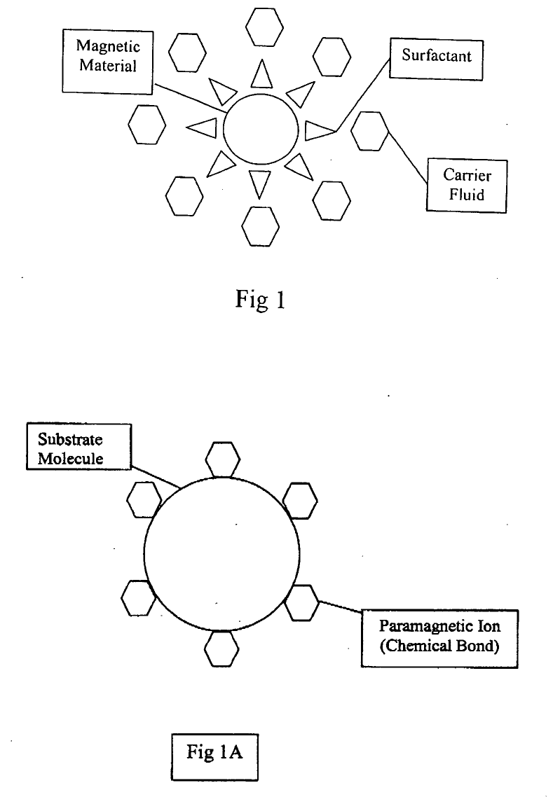

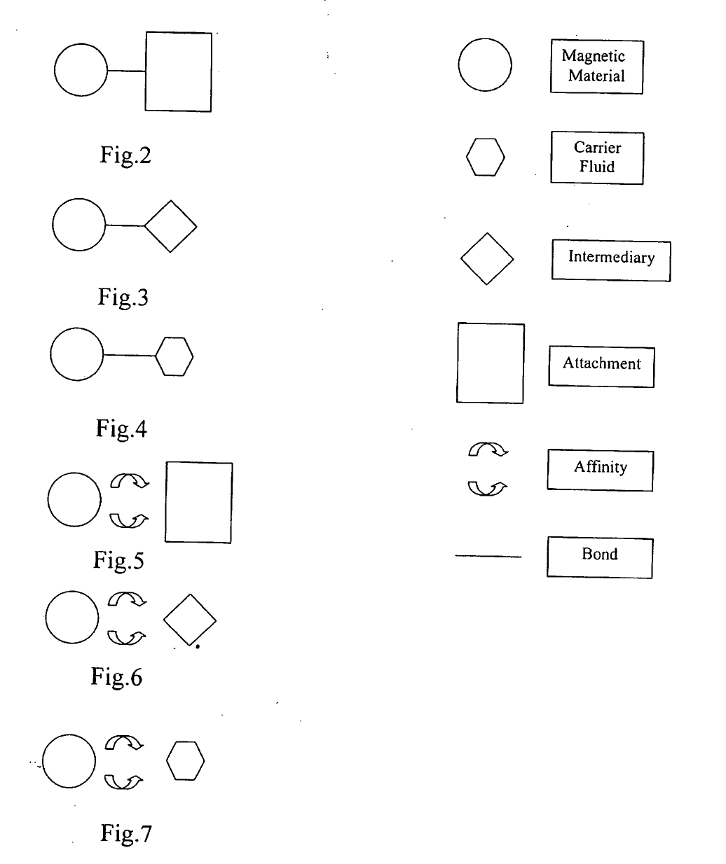

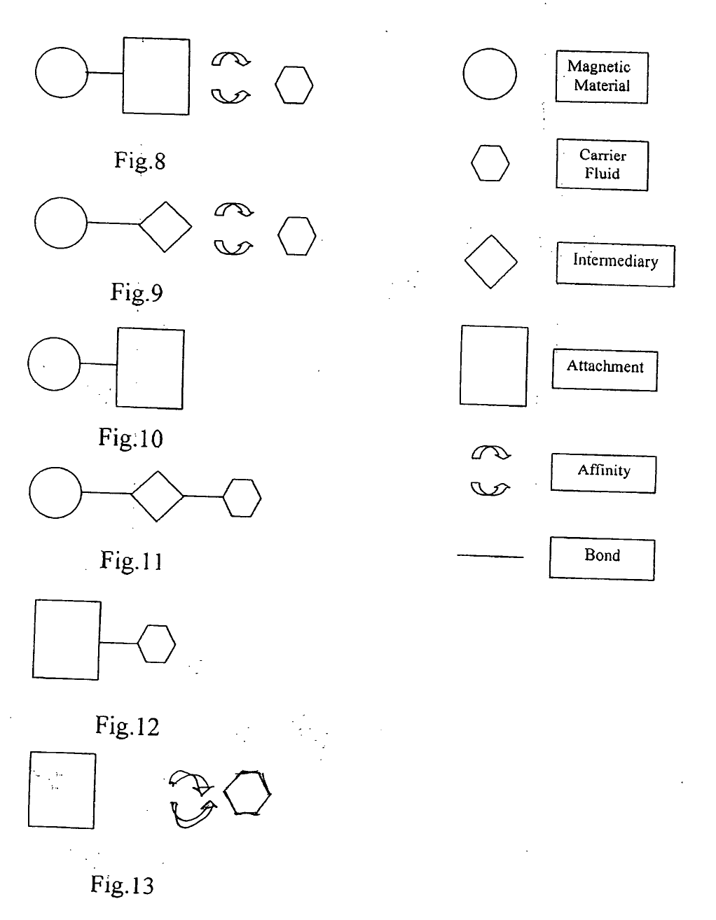

A PVES, as that term is used herein, is a liquid having magnetic properties so as to be attracted by magnets, preferably but not necessarily having rheologic properties. The magnetic properties can be via particles of magnetic material in suspension, or a paramagnetic ion or compound attached to a substrate molecule. FIG. 1A depicts a PVES in a preferred form with its basic components, paramagnetic ions bonded to a substrate (carrier) molecule. Any fluid that can be attracted, or controlled in any manner by a magnetic field can be used.

FIG. 1 shows a specific embodiment wherein the magnetic material is suspended in a carrier fluid, typically with a surfactant coating the magnetic material to help prevent aggregation of the magnetic material. FIG. 1 depicts a PVES of this embodiment with its basic components. The magnetic material is typically coated with a surfactant suspended in the carrier fluid. The rheologic fluid or PVES is not limited to any particular material. As above, any fluid that can be attracted or controlled (especially, gathered) in any manner by a magnetic field can be used.

In one example, the compounds of the PVES in FIG. 1 can be hyaluronic acid as the carrier fluid, and iron oxide or gadolinium oxide as the paramagnetic material. The surfactant can be one of many candidates of molecules from a group of water soluable polysaccharides. Others could be core-shell crosslinked, water soluable polymers, core-shell particles stabilized with amino dextran, hydrophobically modified hydroxyethyl cellulose, acetylene glycol non-ionic surfactants and/or any other surfactant known to polymer chemists that work in a water based environment.

There are multiple embodiments of PVES depending on the exact relationship between the components. The components include: paramagnetic ion or material, carrier fluid, intermediary, attachment. They can be associated by bonds, affinities, or other connection. Attachments and intermediaries can be ions, atoms, molecules, and/or compounds organic or inorganic. They can also include structures such as liposome or complex cellular forms, natural or synthetic. Attachments can be substances that make the magnetic material more compatible biologically and/or more compatible within the PVES. Attachments can also have characteristics that allow them to provide additional functional or therapeutic effects to the PVES besides rheological functions.

Attachments can bind with metal ions or particles from metal/metal wear or UHMWPE wear debris particles to sequester them and inhibit their migration throughout the joint, with the PVES being retained in place by a magnetic field. Attachments can be bound to the magnetic material (FIG. 2) or have an affinity to the magnetic material (FIG. 5). An organic chemist skilled in the art of organo-metallic compounds is familiar with the chemical reactions involved herein, and the sequencing of reactions to add metal ions or compounds to organic materials. The many textbooks on the subject include Supramolecular Organometallic Chemistry by Ionel Haiduc and Frank Thomas Edelmann, Handbook of Inorganic and Organometallic Chemistry by Gmelin, Inorganic and Organometallic Reaction Mechanisms, 2^{nd} Edition by Jim D. Atwood. As mentioned above, hyaluronic acid is an example of a carrier fluid, one which is biocompatible. Hyaluronic acid (HA) is a polymer of glucuronic acid and glucosomine and can have variable lengths up to a few million daltons. There are hydroxyl groups, carboxyl groups and amide groups that are free to be used in chemical reactions. The repetitive groups provide almost unlimited sites for chemical interaction.

Sites can be blocked by various chemical reactions and metal ions added selectively. Metal ions are chosen for their ability to be influenced by a magnetic source. Iron and the lanthanide metals (neodymium, gadolinium, etc.) are the preferred metals and ions. Different amounts of different metal ions and compounds can be added to a single polymer chain affecting the properties of the HA-metal ion compound. The final fluid can be a blend of different proportions of different HA-metal ion molecules giving the final fluid different characteristics, especially in relation to viscosity when influenced by a magnetic field.

Intermediaries act somewhat differently than attachments though they can function as both. Intermediaries help or enhance the PVES function in the particular application. They can help change viscosity of the carrier fluid, prevent the PVES from dispersing or assist in its re-accumulation. Intermediaries can be bound to the magnetic material (FIG. 3) or have an affinity for the magnetic material (FIG. 6). Intermediaries, attachments, affinities and bonds are well within the general knowledge of those skilled in the art of organic chemistry and organo-metallic compounds. In the case of a suspension as the PVES, intermediaries can help suspend the magnetic material, prevent clumping of the magnetic material, change viscosity of the carrier fluid, prevent the fluid from dispersing or assist in its re-accumulation.

Paramagnetic ions or magnetic material can bond (FIG. 4) or have an affinity (FIG. 7) directly with the carrier fluid/substance.

More complex interactions of components in the PVES are shown in FIGS. 8-11. Attachments can bond (FIG. 12) or have an affinity (FIG. 13) for the carrier fluid or substrate. This is not a complete list of interactions between components of PVES and does not limit the scope of PVES that can be used in the applications of animal and mammalian joints or joint replacement.

The PVES or fluid can be placed directly in the desired area, or it can be placed near that area and then manipulated to the desired area by an applied magnetic field. The fluid can be injected into a joint, tissue, organ, fracture site or body part. Another method is to make an incision and then insert the fluid in or near the desired area.

As the carrier fluid, any natural or synthetic biocompatible liquid can be used, with a suspension (for the FIG. 1 embodiment) of paramagnetic particles (iron, lanthanide metals and/or any other paramagnetic solid). In most preferred embodiments of the invention, such a fluid is capable of acting as a lubricant in a joint, and can be a viscous liquid, gel, hydrogel, or, specifically, hyaluronic acid. In the case of a suspension (as in FIG. 1), the particle size for the magnetic particles is in the range of about 0.01 to 100 nm, but preferably is 10 nm or less, as particles of this size are less likely to provoke an inflammatory response from the body and will cause less third-body wear.

In the case of ionic paramagnetic material (FIG. 1A), any natural or synthetic biocompatible liquid can be used as a carrier liquid molecule, with a direct attachment of paramagnetic ions or compounds (of metals as noted above). The magnetic material can include iron or iron compounds, lanthanide metals or compounds of lanthanide metals, or any other paramagnetic substance or compound of a paramagnetic substance.

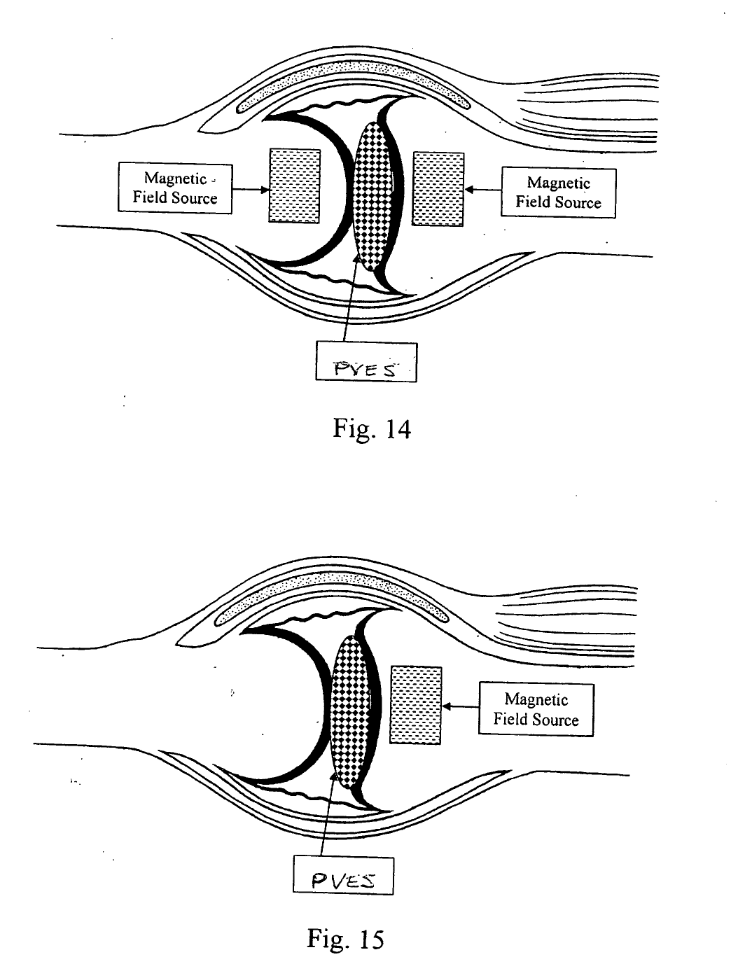

FIG. 14 shows a representative animal or mammalian joint with more than one magnetic source. This is a diarthrodial joint but the joint can be any type of joint. The paramagnetic visco-viscoelastic supplement is designated as PVES in this and the following figures. These figures represent particular embodiments but the indicated PVES is not meant to restrict the type of PVES to a specific type of molecule and not necessarily to require rheologic liquid. The magnetic sources activate the PVES and substantially help maintain it in position. Multiple magnetic field sources can be used to provide greater control of the PVES. A simple single source can be used (FIG. 15).

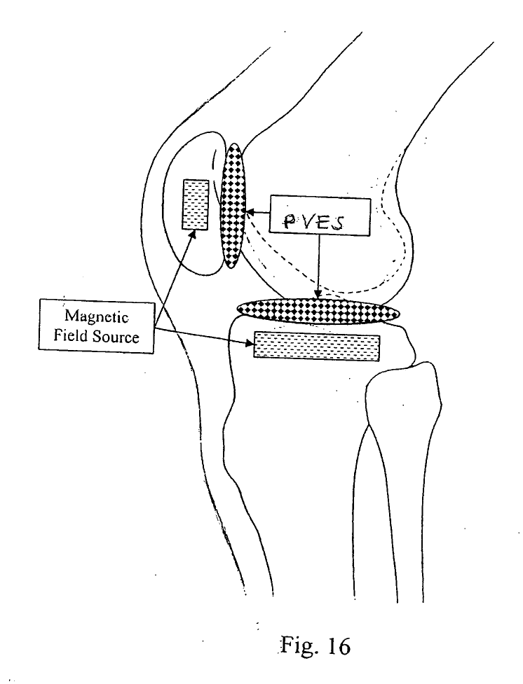

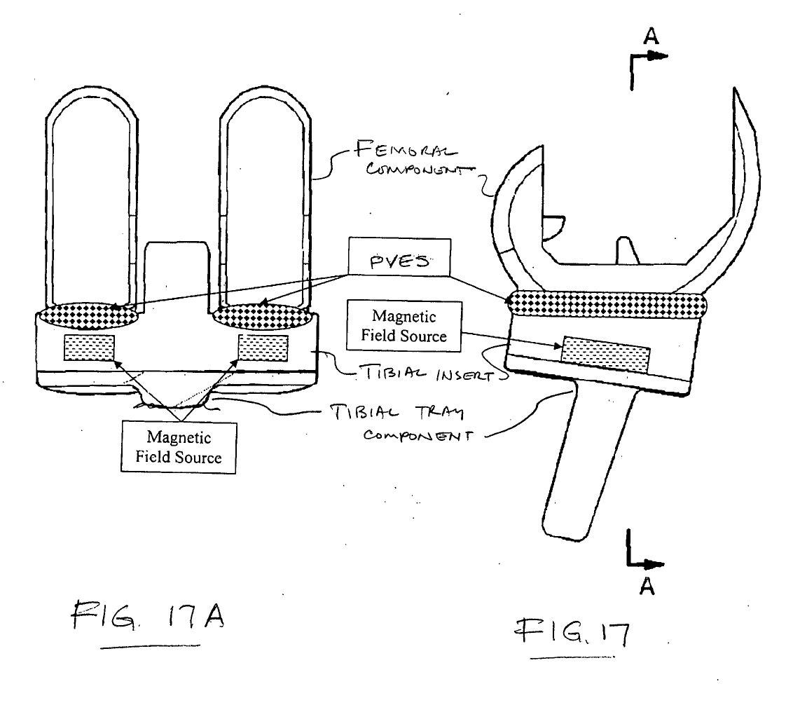

FIG. 16 shows a lateral view of a human knee. In this figure magnetic field sources are placed to collect the PVES at three different areas (medial compartment not shown) in the tri-compartmental knee. Any one or all of the compartments can be enhanced by a PVES. The patellofemoral articulation can be addressed separately for pathologies localized to that joint only. For the patella, for example, the surgeon can open the skin, drill a hole in the patella and insert a permanent magnet, secured by cement, fasteners or surface treatments. An example of a preferred type of permanent magnet is a NeFeB magnet. Other sources can be electromagnets placed externally, or materials that can be magnetized and demagnetized. Magnetic field sources can be held in place externally on the body in a device such as a belt or brace.

Total knee replacements (TKA) can have a PVES between the articulations of the components. FIG. 17 demonstrates the PVES between the femoral and tibial components. The PVES can also be used between the patella and the femoral component. This can be with the use of a patellar resurfacing component or with the natural patella.

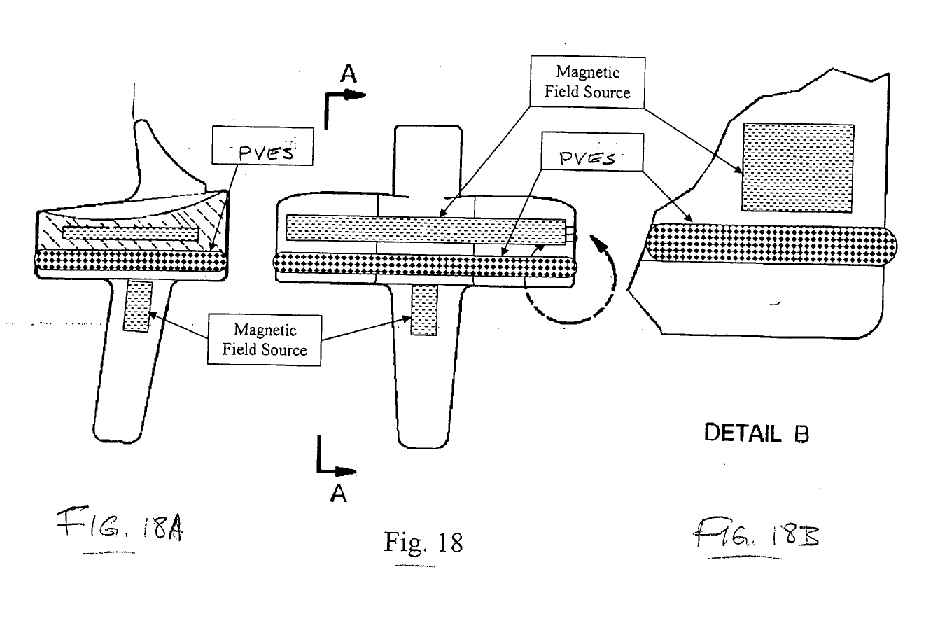

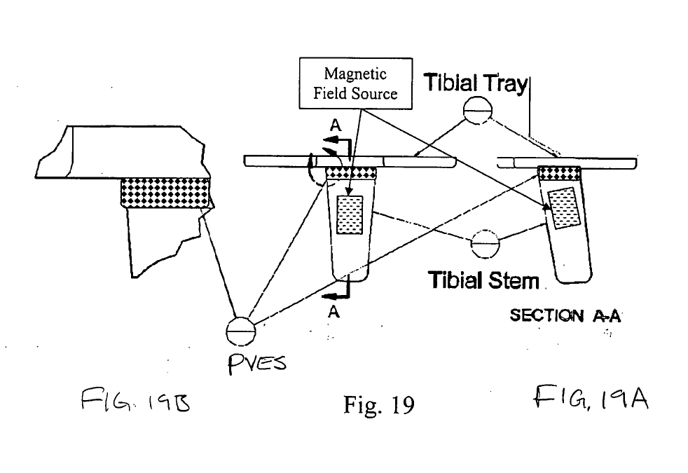

Inter-component functions of the PVES are demonstrated in FIGS. 18-18B and 19-19B. The PVES (FIG. 18) is shown between the tibial insert and the tibial tray. This can be used in both fixed-bearing and mobile-bearing tibial components. The PVES reduces or eliminates back-side wear of the polyethylene in tibial components with tibial inserts that are not rigidly or adequately fixated to the tibial tray. The PVES also allows the tibial insert to be designed so that it moves with respect to the tibial tray but still having a constraint, which is dynamic rather than static.

The PVES or fluid interface (FIGS. 19-19B) is shown between part of a modular tibial tray component. This tibial tray component has a separate tray and stem. The PVES acts as a dynamic interface between the actual tray and the stem.

The PVES can be used in more than one level in a component such as a combination of FIGS. 18 and 19.

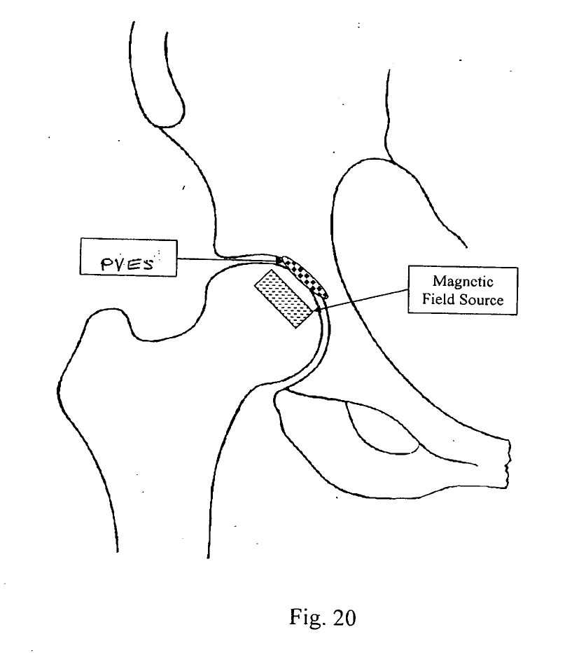

The PVES (FIG. 20) is shown used in the hip joint with one magnetic field source. Multiple field sources or a magnetic array can be used.

The PVES can also be used between hip prosthesis components or between modular component parts. The PVES can be used between the head and the stem, acetabular cup and liner, etc.

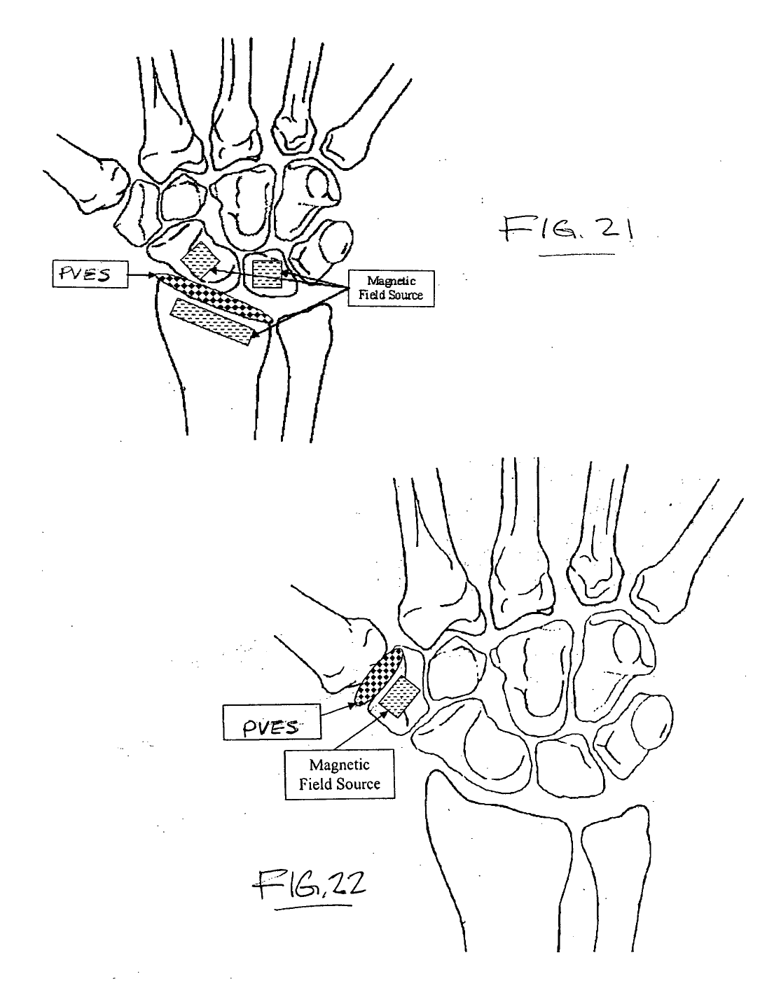

The PVES can be used in small joint applications, FIG. 21 and FIG. 22. FIG. 21 shows the PVES used at the radiocarpal joint without a prosthesis. The PVES can be used between components or between component parts. FIG. 22 shows the PVES at the first metacarpal-carpal joint.

The magnetic field source can be implanted permanently or it can be removed. Superparamagnetic materials such as permadur can be magnetized in situ by an applied magnetic field.

The preferred embodiment for a joint or TJR uses permanent NdFeB or other types of magnets implanted in, behind or near a joint, joint component or components. Magnetic fields are not disturbed by the non-ferromagnetic materials used in the TJR superstructure.

The magnets can be placed in association with one or more of the joint components. The permanent magnets attract the PVES to the desired position as well as activate the properties of the fluid. The fields help maintain the fluid in position and act to re-accumulate dispersed fluids back to their optimal position as they are circulated through the joint space as it moves. The field can have the additional effect altering the viscosity of the fluid if it is rheomagnetic.

Another embodiment uses electromagnets that are activated to respond to certain positions or forces applied across the joints. That is, the field can influence a material only in certain joint positions or under certain joint forces, such as turning on/off an electromagnet to supply the field at a desire pressure, angle at joint, load, impact, etc.

Another embodiment uses combinations of magnets and electromagnets.

Another embodiment uses magnetic induction to produce the magnetic fields and currents in the components themselves or in coils that have magnetic fields and currents induced by the motion of the permanent magnetic material.

Another embodiment uses an electric current to PVES as antibacterial or to change physical properties of the PVES.

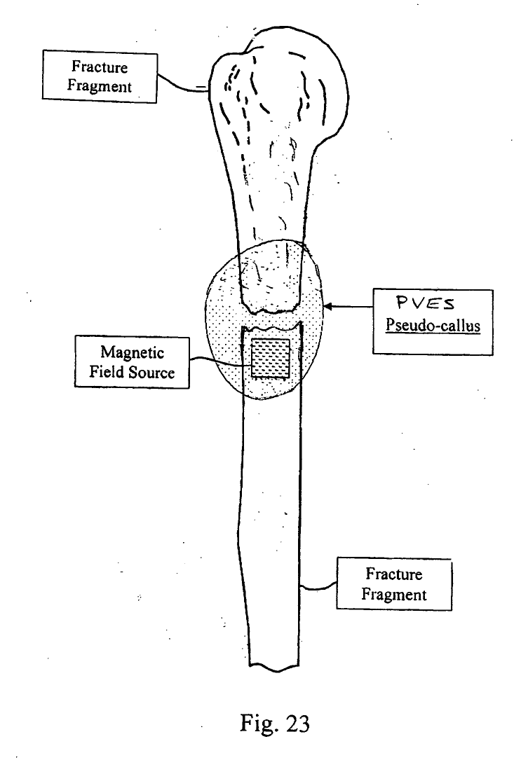

The PVES or fluid interface can be used to structurally augment bone fragments or parts by being introduced directly in the fracture hematoma or immature fracture callus, as in FIG. 23. The magnetic field source (e.g. a permanent magnet) can be implanted permanently or it can be removed. Superparamagnetic materials such as permadur can be magnetized in situ by an applied magnetic field. The PVES substantially adds mechanical support to the bone fragments. It makes them easier to control during reduction and helps maintain them in the preferred position. The PVES can also be used to maintain and/or concentrate additional substances in the fracture environment, such as pseudo-callus, a collection of blood and body products that will eventually become bone. The substances can include platelets, calcium, lattice, etc., anything which has been found to aid in bone healing at a fracture. The magnetic field retains the PVES with the desired substances in place for a useful duration of time.

The preferred embodiment for fracture uses permanent NdFeB or other types of magnets implanted in, behind or near the fracture. Magnetic fields source can be implanted or applied externally. Magnetic field sources can be activated before implantation or in situ if a ferromagnetic or like material is used that can be magnetized. Ferromagnetic material that has a reasonably low coercivity that can be magnetized or degaussed after it is implanted can be used. Paramagnetic material can be magnetized temporarily by an applied field. Other embodiments use non-newtonian fluids especially dilatants to damp forces as the dilatants become more viscous as the shear increases.

Particle size is important in the fluid interface or PVES between surfaces that articulate because it is imperative not to increase wear due to the particles in the fluid interface. The typically small size (10 nm) and the coatings and the dilution of the magnetic material makes their contribution to wear insignificant if there is any contribution at all. Particle size on the PVES in fracture treatment is not limited by this restriction to very small size particles, magnetic or otherwise. Substances that provide structure, structural lattice, and structural components of any reasonable size can be included in the PVES. Substances that are known to promote bone healing can be included such as bone morphogenic protein (BMP), hydroxyapatite, bone graft, synthetic superstructure or matrix. Other fibers, filaments, crystals, rods (carbon-nanotubules) can be included in the fluid interface.

Multiple magnets are used in some situations, including, as shown in the drawings, situations where a PVES or rheologic fluid is to be contained at multiple locations. Another application for multiple magnets is to shape a fluid interface or PVES, e.g. into a circle, oval, toroid or other shape. Multiple magnets may be beneficial in some situations simply to increase the magnetic field. Another situation where multiple magnetic fields can be applied is to have repulsing magnetic fields on either side of a joint, such fields acting as magnetic arrays as in U.S. Pat. No. 6,387,096, referenced above. Such magnets in addition will influence and localize a fluid interface or PVES, using only the attraction function.

Some terms used herein and in the claims are to be understood in a broad sense. “Patient” refers to human or animal patients, and “joints” refer to those of humans or animals. “PVES” is to be understood as a paramagnetic substance whether or not with rheologic properties. “Chemically bonded to” includes direct bonds and indirect bonds, as with an intermediary.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

I claim:

1. A method to improve the lubrication and comfort in joints, at an interface between bones or between synthetic supporting components or between a bone and a synthetic supporting component in the joint of a patient, comprising:
placing at least one magnet field source in, on or around the bones or synthetic components adjacent to the interface,
placing into or near a space at the interface in the joint, a fluid substance that can be influenced by a magnetic field so as to be atracted by the at least one magnetic field source to tend to collect, remain and to return to accumulated, gathered-together condition at the interface,
whereby the fluid substance tends to collect in and to remain accumulated in the joint and in position to lubricate the interface in the joint, under influence of the at least one magnetic field source, and when forces caused by use of the joint cause the fluid substance to move partially out of the interface, the magnetic field source will return the fluid substance essentially to the same accumulated condition when such forces subside.

2. The method of claim 1, wherein the fluid substance comprises a viscous liquid with therapeutic lubricating properties, and wherein a paramagnetic material is contained within the liquid.

3. The method of claim 2, wherein the paramagnetic material comprises ions or compounds of metal, bonded chemically in the viscous liquid.

4. The method of claim 2, wherein the paramagnetic material comprises small particles in suspension within the viscous liquid.

5. The method of claim 2, wherein the viscous liquid comprises a rheologic liquid so as to have energy-absorbing as well as lubricating properties.

6. The method of claim 5, wherein the rheologic liquid comprises hyaluronic acid.

7. The method of claim 2, wherein the liquid includes a pharmacological material.

8. The method of claim 1, wherein the fluid substance includes paramagnetic material, as well as a therapeutic material.

9. The method of claim 8, wherein the fluid substance comprises hyaluronic acid, serving as a carrier for the paramagnetic material and as a therapeutic lubricant for the joint.

10. The method of claim 8, wherein the therapeutic material comprises a pharmacologic material.

11. The method of claim 1, wherein the fluid substance comprises a liquid, and including particles of paramagnetic material retained in suspension in the liquid.

12. A medical procedure for concentrating and retaining a fluid substance in a region of interest in the body of a human or animal where the fluid substance will have a therapeutic effect, comprising:
placing the fluid substance into or near the region of interest, the substance having paramagnetic properties such as to be attracted by a magnetic field, and
emplacing at least one magnetic field source into or onto the patient, adjacent to the region of interest, in a position so as to impose a magnetic field at the region of interest so that the fluid substance tends to accumulate and remain in the region of interest, and to return to the region of interest if displaced.

13. The medical procedure of claim 12, wherein the magnetic field source is implanted into a bone adjacent to a joint of the patient.

14. The medical procedure of claim 13, wherein the fluid substance comprises a rheologic fluid having lubricating properties to lubricate the joint.

15. The medical procedure of claim 13, wherein the fluid substance includes a pharmacological material.

16. The medical procedure of claim 12, wherein the fluid substance comprises hyaluronic acid, and including particles of iron or lanthanide metal or compounds thereof suspended in the hyaluronic acid, with a surfactant acting on the particles and the hyaluronic acid and retaining the particles in suspension.

17. The medical procedure of claim 16, wherein the metal particles have a particle size no greater than about 10 nanometers.

18. The medical procedure of claim 12, wherein the fluid substance comprises hyaluronic acid, with ions of iron or lanthanide metal or compounds of iron or lanthanide metal bonded to the hyaluronic acid.

19. The medical procedure of claim 12, wherein the fluid substance having paramagnetic properties includes particles of paramagnetic material in suspension in liquid.

20. The medical procedure of claim 19, wherein the paramagnetic material comprises iron, an iron compound, a lanthanide metal or a lanthanide metal/compound.

21. The medical procedure of claim 20, wherein the particles are of a size no larger than about 10 nanometers.

22. The medical procedure of claim 12, wherein the fluid substance includes an analgesic or antibiotic.

23. The medical procedure of claim 12, wherein the region of interest comprises a joint and-wherein the fluid substance comprises a rheologic liquid having lubricating effects for the joint and also including a therapeutic drug carried with the rheologic liquid.

24. The medical procedure of claim 23, wherein the liquid includes paramagnetic particles in suspension.

25. The medical procedure of claim 23, wherein the liquid includes metal ions or compounds chemically bonded to the liquid.

26. The medical procedure of claim 12, wherein the fluid substance is influenced by a magnetic field, not only to tend to accumulate and collect the liquid substance in the region of interest, but also in another way which changes a property of the fluid substance.

27. The medical procedure of claim 26, wherein the property changed by the magnetic field comprises viscosity, shape of fluid, molecular configuration, liquid density, or storage of potential energy.

28. The medical procedure of claim 12, wherein the step of placing the fluid substance comprises placing the substance at a position spaced away from the region of interest, and drawing the substance into the region of interest with the magnetic field.

29. A fluid substance comprising a lubricant combined with a paramagnetic material in a manner that prevents settling or separation of the paramagnetic material from the lubricant, whereby the fluid substance can be affected by a magnetic field, such that the application of a magnetic field can exert a force on the fluid substance or change a property of the fluid substance.

30. The fluid substance of claim 29, wherein the fluid substance is a liquid and the paramagnetic material comprises small particles retained in suspension in the liquid, and including a surfactant retaining the particles in suspension in the lubricant liquid.

31. The fluid substance of claim 30, wherein the paramagnetic particles have a particle size in the range of about 0.001 nm to 1000 nm.

32. The fluid substance of claim 31, wherein the particles have a particle size in the range of about 1 nm to 10 nm.

33. The fluid substance of claim 29, wherein the paramagnetic material comprises metallic ions or compounds chemically bonded to molecules of the lubricating liquid.

34. The fluid substance of claim 29, wherein the lubricant comprises hyaluronic acid, cross-linked hyaluronic acid, gel, or hydrogel.

35. The fluid substance of claim 29, wherein the lubricant is a visco-elastic liquid.
